(19) United States
(12) Patent Application Publication
(10) Pub. No.: US 2024/0150777 A1
(43) Pub. Date: May 9, 2024

(54) RADIO FREQUENCY RESPONSIVE PLANT EXPRESSION VECTOR

(71) Applicant: BIO-FD&C, Incheon (KR)

(72) Inventors: Sang Hyun MOH, Seongnam-si (KR); Jeonghun LEE, Seoul (KR); Young Soon KIM, Gwangju (KR); Sunmee CHOI, Seoul (KR); Soo-Yun KIM, Incheon (KR); Giseok KWON, Yongin-si (KR); Hyein KIM, Incheon (KR); Ga-Eun BAEK, Incheon (KR); Suk Hyun KWON, Incheon (KR); Jiyeon KIM, Siheung-si (KR); Seungjun LEE, Incheon (KR)

(73) Assignee: BIO-FD&C, Incheon (KR)

(21) Appl. No.: 18/505,525

(22) Filed: Nov. 9, 2023

(30) Foreign Application Priority Data
Nov. 9, 2022 (KR) 10-2022-0148692

(51) Int. Cl. C12N 15/82; C12N 5/04; C12N 9/88; C12P 21/02

(52) U.S. Cl. CPC C12N 15/8237; C12N 5/04; C12N 9/88; C12N 15/8205; C12N 15/8251; C12P 21/02; C12Y 402/0102; C12N 2830/002

(57) ABSTRACT

The present disclosure relates to a recombinant vector for plant expression system in which expression is increased by a radio frequency. According to the present disclosure, since the recombinant vector for plant expression of the present disclosure significantly increased the expression of the target protein in plants by treatment of an eco-friendly and biologically stable radio frequency, there is an effect of overcoming a low protein expression problem of a method using plant cells while having an advantageous effect capable of replacing a conventional production method using animal cells or microorganisms.

### CROSS-REFERENCE TO RELATED APPLICATIONS

This application is based on and claims priority from Korean Patent Application No. 10-2022-014692, filed on Nov. 9, 2022, with the Korean Intellectual Property Office, the disclosure of which is incorporated herein in its entirety by reference.

### INCORPORATION BY REFERENCE OF SEQUENCE LISTING

The content of the electronically submitted sequence listing, file name: Q291642_sequence listing as filed; size: 7,276 bytes; and date of creation: Nov. 9, 2023, filed herewith, is incorporated herein by reference in its entirety.

### TECHNICAL FIELD

The present disclosure relates to a recombinant vector system for plant expression in which expression is increased by radio frequency.

### BACKGROUND

Production of recombinant proteins includes methods using microorganisms, animals, or plants. Traditionally, the production of recombinant proteins using microorganisms or animal cells has been widely performed. However, recombinant proteins produced primarily from animal cells or microorganisms have the disadvantage of increasing product prices due to high purification costs and additional facility investment costs during commercialization. In addition, when using microbial systems to produce recombinant proteins, there are cases where the protein's activity does not appear due to lack of modification after protein expression. Also, when using animal cells to produce recombinant proteins, there is a risk of infection by viruses or pathogens. To compensate for these shortcomings, research on producing recombinant proteins using plant expression systems has been actively conducted in recent years.

In general, the production of useful bioactive substances using transgenic plants may fundamentally exclude various contaminants, such as viruses, cancer genes, and enterotoxins that may occur in a method of producing proteins synthesized in animal cells or microorganisms. Since the plant expression system is absolutely advantageous compared to existing animal cell systems in terms of equipment technology and cost required for mass production when the demand for the useful substances increases rapidly. Plant expression systems have revolutionized various research fields, offering cost-effective and rapid solutions for protein production. They play a pivotal role in basic science research, vaccine development, and therapeutic development. As technology continues to advance, plant-based systems are likely to become even more critical in addressing scientific and medical challenges while ensuring affordability and accessibility of important bioproducts.

The production of bioactive substances from plants has the potential to replace traditional methods utilizing animal cells or microorganisms for several compelling reasons. Firstly, the time required for producing valuable bioactive substances can be significantly reduced, leading to substantial cost savings. Secondly, the risk of contaminants, such as cytotoxicity, often encountered during the separation and purification of proteins derived from animal cells or microorganisms, can be eliminated. Thirdly, bioactive substances can be stored for extended periods in the form of plants or seeds, reducing expenses associated with preservation and storage. Fourthly, bioactive substances in seed form can be safely transported and quickly supplied to regions in cases of urgency. Lastly, mass production of bioactive substances becomes more feasible due to the simpler technology requirements for building production facilities and systems. In comparison to animal cell systems, this approach dramatically reduces both facility and product production costs, ensuring a rapid and ample supply in response to market demand.

In spite of these advantages, protein production in plant cells faces challenges due to relatively lower protein expression levels and less optimized isolation and purification methods compared to alternative hosts like animal cells and microorganisms. Consequently, extensive research efforts have been dedicated to enhancing protein expression and productivity within plant cells using various strategies. This includes the development of vector construction technology to efficiently introduce foreign genes into plants and boost the expression of target proteins, as well as advances in plant transformation techniques and protein isolation and purification technologies for the production of target proteins within transgenic plants.

To ensure the competitiveness of plant-based platforms for recombinant protein production, it is imperative to enhance productivity by optimizing processes, such as increasing protein expression and reducing production times. Plants employ an induction system that governs gene transcription in response to a range of stimuli, including light, temperature, hormones, chemicals, stress, and physical injury. These inducers typically elicit pleiotropic effects in plants through intricate signal transduction pathways, making their practical application and monitoring a complex task. Hence, this innovation seeks to utilize physical inducers as a straightforward, harmless, and temporary method to stimulate plant responses. In this context, our objective is to elevate the expression of target proteins by subjecting plants to radiofrequency treatment. Our approach also involves the identification of promoters responsive to radiofrequency and leveraging them to enhance the expression of target genes.

### SUMMARY

The present disclosure has been made in an effort to provide an inducible promoter that increases the expression of a target protein by a radio frequency.

The present disclosure has also been made in an effort to provide a recombinant vector for plant expression of a target protein.

The present disclosure has also been made in an effort to provide Agrobacteria transformed with a recombinant vector for plant expression.

The present disclosure has also been made in an effort to provide a plant transformant.

The present disclosure has also been made in an effort to provide a composition for plant transformation.

The present disclosure has also been made in an effort to provide a method for enhancing a target protein.

In order to achieve the above objects, an embodiment of the present disclosure provides an inducible promoter that increases the expression of a target protein by a radio frequency.

Another embodiment of the present disclosure provides a recombinant vector for plant expression including a CsVMV promoter of the present disclosure and a nucleic acid encoding a target protein.

Yet another embodiment of the present disclosure provides Agrobacteria transformed with a recombinant vector for plant expression.

Yet another embodiment of the present disclosure provides a plant transformant for producing a target protein.

Yet another embodiment of the present disclosure provides a composition for plant transformation.

Yet another embodiment of the present disclosure provides a method for producing a target protein in plants.

According to the embodiments of the present disclosure, an inducible promoter responding to eco-friendly and biologically stable RF was developed, and a plant expression vector combining the developed promoter and a target protein expression module was constructed to enable high expression of the target protein in conjunction with RF treatment. Addressing the challenge of low protein expression in plant cells offers a significant advantage as it allows for the substitution of traditional production techniques involving animal cells or microorganisms. This improvement leads to enhanced protein synthesis, resulting in increased yields of recombinant proteins and a shortened production timeline. Consequently, the development of a plant-based production platform that consistently augments recombinant protein output, without impeding regular plant growth, holds substantial commercial promise.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

FIG. 1 is a schematic diagram of a recombinant protein expression module in the vector of the present disclosure:
A: Vectors for verification of protoplast transformation (PlantGEM-V2-1: a control vector containing TS-α promoter; PlantGEM-V2-5: a vector containing CaMV35S promoter; and PlantGEM-V2-7: a vector containing CsVMV promoter); and
B: Vectors used for Agrobacteria infiltration (PlantGEM-V3-5: a vector containing CaMV35S promoter; and PlantGEM-V3-7: a vector containing CsVMV promoter).

FIG. 2 is a diagram confirming an operation of a promoter by transforming protoplasts of a plant (tobacco).

FIG. 3 is a diagram showing a process of infiltrating Agrobacteria into tobacco leaves using a syringe (syringe agroinfiltration).

FIG. 4 is a diagram showing a method of treating a radio frequency to pots in which tobacco plants infiltrated with Agrobacteria are planted.

FIG. 5 is a diagram of confirming an expression result of a GFP protein in plants infiltrated by Agrobacteria containing a PlantGEM-V3-5 vector or PlantGEM-V3-7 vector after radio frequency treatment by a fluorescence microscopy image:
DAI: Number of days after Agrobacteria infiltration.
NT: Radio frequency-untreated group; and
RF: Radio frequency-treated group.

FIG. 6 is a diagram of confirming GFP expression in plants infiltrated by Agrobacteria containing a PlantGEM-V3-5 vector or PlantGEM-V3-7 vector during radio frequency treatment by Western blot assay:
DAI: Number of days after Agrobacteria infiltration.
NT: Radio frequency-untreated group; and
RF: Radio frequency-treated group.

FIG. 7 is a graph of quantifying a Western blot assay result of GFP expression in plants infiltrated by Agrobacteria containing a PlantGEM-V3-5 vector or PlantGEM-V3-7 vector after radio frequency treatment.

FIG. 8 is a diagram of confirming expression results of GFP protein by frequency (170, 260, 360, 560 and 630 kHz) using PlantGEM-V3-7 by fluorescence microscope images.

FIG. 9 is a diagram of confirming the expression of GFP protein by frequency (170, 260, 360, 560 and 630 kHz) using PlantGEM-V3-7 by Western blot assay.

FIG. 10 is a diagram of quantifying a Western blot assay result of GFP protein by frequency (170, 260, 360, 560 and 630 kHz) using PlantGEM-V3-7.

FIG. 11 is a diagram of confirming the expression results of GFP protein of PlantGEM-V3-7 according to an RF treatment time at a frequency of 360 kHz by fluorescence microscope images.

FIG. 12 is a diagram of confirming the expression level of GFP protein of PlantGEM-V3-7 according to an RF treatment time at a frequency of 360 kHz by Western blot assay.

FIG. 13 is a diagram of quantifying a Western blot assay result of GFP protein of PlantGEM-V3-7 according to an RF treatment time at a frequency of 360 kHz.

FIG. 14 is a diagram of confirming the radio-frequency waveforms generated in Agroinfiltrated tobacco leaves during RF treatment based on the processing frequency. At 170 kHz, the waveform appears distorted due to higher harmonic resonance, and the large and small waveforms are one cycle.

### DETAILED DESCRIPTION

In the following detailed description, reference is made to the accompanying drawing, which forms a part hereof. The illustrative embodiments described in the detailed description, drawing, and claims are not meant to be limiting. Other embodiments may be utilized, and other changes may be made, without departing from the spirit or scope of the subject matter presented here.

Hereinafter, an embodiment of the present disclosure will be described in detail with reference to the accompanying drawings. However, the following embodiments are presented as examples for the present disclosure, and when it is determined that a detailed description of well-known technologies or configurations known to those skilled in the art may unnecessarily obscure the gist of the present disclosure, the detailed description thereof may be omitted, and the present disclosure is not limited thereto. Various modifications and applications of the present disclosure are possible within the description of claims to be described below and the equivalent scope interpreted therefrom.

Terminologies used herein are terminologies used to properly express preferred embodiments of the present disclosure, which may vary according to a user, an operator's intention, or customs in the art to which the present disclosure pertains. Therefore, these terminologies used herein will be defined based on the contents throughout the specification. Throughout the specification, unless explicitly described to the contrary, when a certain part “comprises” a certain component, it will be understood to imply the inclusion of stated elements but not the exclusion of any other elements.

All technical terms used in the present disclosure, unless otherwise defined, have the meaning as commonly understood by those skilled in the related art of the present disclosure. In addition, although preferred methods and samples are described herein, similar or equivalent methods and samples thereto are also included in the scope of the present disclosure. The contents of all publications disclosed as references in this specification are incorporated in the present disclosure.

In one aspect, the present disclosure relates to a promoter including a nucleotide sequence represented by SEQ ID NO: 1.

In one embodiment, the promoter may be a CsVMV promoter or an inducible promoter that increases the expression of a target protein by radio frequency treatment.

In one embodiment, the present disclosure relates to an expression cassette including the promoter of the present disclosure, a nucleic acid encoding a target protein, and a Nos terminator (Tnos).

In one aspect, the present disclosure relates to a recombinant vector for plant expression including the expression cassette of the present disclosure.

In one embodiment, the cassette may be inserted into a pGEM vector.

In one embodiment, the vector may further comprise a coding sequence of plant selection markers genes, the genes preferably are antibiotic resistance genes or herbicide resistance genes.

In one embodiment, the cassette may be inserted into a binary vector.

In the present disclosure, the “binary vector” refers to a standard tool in the transformation of higher plants mediated by Agrobacterium tumefaciens. It is preferably composed of the borders of T-DNA, multiple cloning sites, replication functions for Escherichia coli and A.

In one embodiment, Tnos may include a nucleotide sequence represented by SEQ ID NO: 2.

In one embodiment, pCaMV35S may include a nucleotide sequence represented by SEQ ID NO: 3.

In one embodiment, HPT may include a nucleotide sequence represented by SEQ ID NO: 4.

In one embodiment, CsVMV may include a nucleotide sequence represented by SEQ ID NO: 5.

In the present disclosure, variants of the sequence are included within the scope of the present disclosure. The variant is a nucleotide sequence having a functional property similar to that of the nucleotide sequence represented by SEQ ID NO shown above, although the nucleotide sequence is changed. Specifically, the promoter may include a nucleotide sequence having sequence homology of 70% or more, 80% or more, or 90%, 91%, 92%, 93%, 94%, 95%, 96%, 97%, 98%, 99% or more of the nucleotide sequence represented by SEQ ID NO: 1. The “% of sequence homology” with a polynucleotide is determined by comparing two optimally arranged sequences with a comparison region, and a part of a polynucleotide sequence in the comparison region may include addition or deletion (i.e., gap) compared to a reference sequence (without including addition or deletion) for an optimal alignment of the two sequences.

In one embodiment, the vector of the present disclosure may increase the expression of a target protein by radio frequency (RF) treatment.

In one embodiment, the vector of the present disclosure may increase the expression of the target protein by radio frequency treatment of 30 KHz to 30 MHz, and more preferably radio frequency treatment of 100 to 700 KHz.

In one embodiment, the vector of the present disclosure may increase the expression of the target protein by radio frequency treatment of 360 kHz.

In one embodiment, the plant may be plant cells, plant culture roots or plant bodies.

In one embodiment, the plants may be plants of Nelumbo nucifera, Acanthopanax senticosus, Artemisia annua, Coriandrum sativum, Sesamum indicum, Sedum kamtschaticum, Silkworm thorn, Ipomoea hederacea, Morinda citrifolia, Daucus carota, Dendrobium moniliforme, Glycine max, Camellia japonica, Helianthus tuberosus, Prunus mume, Polygonum Fagopyrum, Lilium candidum, Malus pumila, Rubus crataegifolius, Panax ginseng, Acorus gramineus, Campanula takesimana, Sequoiadendron giganteum, Damnacanthus major, Brassica rapa, Angelica keiskei, Oryza sativa, Ginkgo biloba, Cucumis sativus, Allium sativum, Echinacea purpurea, Althaea rosea, Perilla frutescens, Acorns calamus, Pelargonium sidoides, Vitis vinifera, Neofinetia falcata, Hypericum androsaemum, Aster sphathulifolius, Rosa rugosa, Secale cereale, Broussonetia kazinoki, Rosa davurica, Leonurus sibiricus, Camellia sinensis, Cnidium officinale, Foeniculum vulgare, Rosmarinus officinalis, Magnolia Kobus, Prunus serrulata, Aloe barbadensis, Opuntia ficus, Cosmos bipinnatus, Dendropanax morbifera, Leontopodium alpinum, Annona muricatu, Psidium Guajava, Tulipa Gesneriana, Narcissus tazetta, Solanum lycopersicum, Artemisia princeps, Vaccinium angustifolium, Morus alba, and the like, and may include all plant cells, plant culture roots or plant bodies.

In one embodiment, the vector of the present disclosure may further include a fluorescent material for detecting the plants, and the fluorescent material may be fluorescent protein, photoprotein, luciferase, fluorescent dye, or time-resolved fluorescence (TRF).

In the present disclosure, the “recombinant” refers to a cell that replicates a heterogonous nucleic acid, expresses the nucleic acid, or expresses a protein encoded by a peptide, a heterogenous peptide or a heterogenous nucleic acid. The recombinant cells may express genes or gene fragments that are not found in a natural form of the cells as either sense or antisense form. The recombinant cells may also express genes found in cells in the natural state, but the genes have been modified and reintroduced into the cells by artificial means.

In the present disclosure, “target protein” refers to a target recombinant protein, and in one embodiment of the present disclosure, as an example of the target protein, GFP is constructed in a vector and only its expression is increased by radio frequency treatment, but it is also obvious to those skilled in the art to use other proteins as the target protein.

Examples of nucleic acids encoding the target protein expressed by the vector of the present disclosure include nucleic acids of genes related to fatty acid biosynthesis and regulation when a plant to be transformed is oilseed crops such as rape, perilla, and sesame, nucleic acids and the like of antibody genes with high economic added value in the case of leguminous plants such as soybean and alfalfa, in which the production and accumulation of proteins are relatively high compared to other plants, nucleic acids and the like of edible vaccine genes such as swine diarrhea and foot-and-mouth disease virus, in the case of corn, which is a representative feed crop, nucleic acids and the like of genes related to resistance to various diseases to increase storage after harvest in the case of most crops that are stored and distributed as seeds for a long time, and nucleic acids and the like of biosynthetic genes of related metabolic pathways in the case of enhancing specific nutritional components, such as tocopherols and beta-carotene, in crop seeds. However, the nucleic acids are not limited thereto, and may include genes (that is, nucleic acids encoding target proteins) expressing various target proteins that may be regulated using a seed-specific promoter of the present disclosure.

In the present disclosure, the “promoter” is a genome region linked to the upper side of a structural gene and serves to regulate the transcription of the structural gene linked thereto into mRNA. The promoter is linked with several general transcription factors to be activated, and generally has nucleotide sequences such as TATA box, CAT box, and the like that regulate gene expression. Proteins necessary for the basic metabolism of the living body need to maintain a certain concentration in the cells, so that the promoter linked to these genes is always active only by the action of general transcription factors. The CsVMV promoter used in the present invention is a constitutive promoter and is always active, so the protein expression rate is good even under normal conditions. However, in the present invention, the promoter was used as a representative radio-frequency response promoter as it was discovered that the protein expression rate was significantly improved in response to radio frequency.

In the present disclosure, the “vector” is used to refer to DNA fragment(s) or nucleic acid molecules that are delivered into cells. The vector replicates DNA and may reproduce independently in host cells. The term “delivery vehicle” is often used interchangeably with “vector”. The term “expression vector” refers to a recombinant DNA molecule containing a target coding sequence and an appropriate nucleic acid sequence necessary to express an operably linked coding sequence in a particular host organism. Promoters, enhancers, termination signals and polyadenylation signals available in eukaryotic cells are known.

In the recombinant expression vector according to one embodiment of the present disclosure, the expression vector may be prepared by operably linking a target gene downstream of the promoter of the present disclosure. In the present disclosure, “operably linked” refers to components of an expression cassette that function as units for expressing a heterogenous protein. For example, a promoter operably linked to heterogenous DNA encoding a protein promotes production of functional mRNA corresponding to the heterogenous DNA.

In the present disclosure, the target gene may be any kind of gene, and for example, genes that encode proteins with medical utility, such as enzymes, hormones, antibodies, and cytokines or encode proteins capable of accumulating a large amount of nutrients that may improve the health of animals, including humans, but is not limited thereto. In addition, the target gene may be a gene existing in a host into which a recombinant plant expression vector is introduced or a foreign gene.

A recombinant expression vector including the promoter of the present disclosure and a nucleotide sequence encoding a target protein operably linked to the promoter may be introduced into plant cells using a method known in the art. As the method for introducing the recombinant expression vector according to the present disclosure into plant cells, any known method may be used without limitation, and for example, there are various methods available for stable or transient genetic transformation, such as electroporation, microinjection, biolistic, PEG mediated DNA uptake, Agrobacterium-mediated, Agro floral dip, Agrobacterium-infiltrated and so on.

In the present disclosure, the “radio frequency” is a wave or radio wave with a high frequency, and although there is no clear definition of frequency, the radio frequency generally refers to a frequency band of 30 kHz to 30 MHz in alternating current (see classification of electromagnetic waves in Table 1 below).

TABLE 1

| | | | Radio frequency | | | | | |
|---|---|---|---|---|---|---|---|---|
| | Low | Medium | Long | Medium | Short | Very high | Ultrahigh | Beam |
| | frequency | frequency | frequency | frequency | frequency | frequency | frequency | radiation |
| Frequency | ~1 KHZ | 2~6 KHz | 30~300 KHz | 300 KHz~ | 3 MHz~ | 30 MHz~ | 3 GHz~ | 300 GHz~ |
| | | | | 3 MHz | 30 MHz | 3 GHz | 300 GHz | |

| | | | | | |
|---|---|---|---|---|---|
| Function | Physical therapy device | Electrosurgical device, AM Radio | FM, TV | Microwave | UV lights, |

etc.

In one aspect, the present disclosure relates to Agrobacteria transformed with the recombinant vector for plant expression of the present disclosure.

In one aspect, the present disclosure relates to a plant transformant that produces a target protein by introducing the recombinant vector for plant expression of the present disclosure or Agrobacteria transformed by the recombinant vector.

In one embodiment, the transformant may be plant culture cells, plant culture roots or plant bodies.

In the present disclosure, the plant transformant is not limited to the type as long as the expression vector is inserted or Agrobacteria are infiltrated. Such plants may include plants themselves, tissues, cells, or seeds.

For example, “plant cells” used for plant transformation may be any plant cell. The plant cell may be cultured cells, cultured tissues, cultured organs or a whole plant, preferably cultured cells, cultured tissues or cultured organs, and more preferably any type of culture cells.

In the present disclosure, the “plant tissue” is differentiated or undifferentiated plant tissues, for example, but is not limited thereto, and includes roots, stems, leaves, pollen, seeds, and various types of cells used in culture, that is, single cells, protoplasts, shoots and callus tissues. The plant tissue may be in planta or may be in an organ culture, tissue culture or cell culture state.

In one aspect, the present disclosure relates to a composition for plant transformation including the vector or Agrobacteria of the present disclosure.

In one aspect, the present disclosure relates to a method for producing a target protein including a) preparing a plant transformant expressing a target protein stably or transiently by introducing the vector of the present disclosure into a plant; b) treating a radio frequency; and c) isolating and purifying the target protein from the transformant.

In one embodiment, the plant may be plant culture cells, plant culture roots or plant bodies.

In one embodiment, the plant transformant in step a) may be prepared by transforming a plant with the vector of the present disclosure.

In one embodiment, the plant transformant in step a) may be prepared by infiltrating a plant with the Agrobacteria of the present disclosure containing the vector of the present disclosure.

In one embodiment, a radio frequency of 30 KHz to 30 MHz may be treated, it is preferable to treat a radio frequency of 100 to 700 KHz, and it is more preferable to treat a radio frequency of 360 kHz.

In one embodiment, the radio frequency may be treated for 1 to 120 minutes, preferably 20 minutes.

Hereinafter, the present disclosure will be described in more detail with reference to the following Examples. However, the following Examples are only intended to embody the contents of the present disclosure, and the present disclosure is not limited thereto.

### Example 1. Construction of Plant-Based Protein Production Platform

1-1. Preparation of Protein Expression Module Vector

To develop a method for dramatically improving plant cell-based recombinant protein production, a plant expression vector containing a constitutive promoter was introduced into plant cells, and a radio frequency (RF) was combined to improve the expression efficiency of the target protein in plants. To this end, vectors, including a protein expression module consisting of a CsVMV promoter (pCsVMV) and a Nos terminator (Tnos) regulating the expression of green fluorescence protein (GFP) as a marker protein, were constructed through a molecular cloning technique. As a control, a CaMV35S promoter was used. Specifically, as a vector for examining gene expression in protoplasts by recombining one expression cassette (pCsVMV: GFP: Tnos) into a pGEM vector, PlantGEM-V2-5 and PlantGEM-V2-7 (FIG. 1A), two cassettes pCsVMV: GFP: Tnos and p35S(enh): HPT: CaMVpolyA were inserted into a pCAMBIA vector for plant transformation using Agrobacteria to prepare PlantGEM-V3-5 and PlantGEM-V3-7 (FIG. 1B).

1-2. Confirmation of Operation of Promoter in Plant Protoplasts

Expression vectors for protoplasts prepared in Example 1-1, PlantGEM-V2-1 (positive control vector containing a promoter pTSα of a Tryptophan synthase alpha-like gene expressed specifically for radio frequency treatment), PlantGEM-V2-5 and PlantGEM-V2-7 were transformed into tobacco protoplasts using a PEG transfection technique, and the operation of the promoter and the protein expression level were confirmed and compared using a fluorescence microscope. Specifically, tobacco (Nicotiana benthamiana) seeds were sowed and grown for 5 weeks in a 16 Light/8 Dark at 25 to 28° C. Then, 3 to 5 young tobacco leaves were cut on a 6 cm petri dish with a thickness of 1 to 2 mm and added in 5 ml of a cellulase solution (Table 2). Thereafter, a green protoplast suspension obtained by incubating for 12 hr or more without stirring in a dark state at 28° C. was filtered through a 70 μm mesh and then transferred to a 15 ml round tube, and 1 to 2 ml of an upper part was taken through centrifugation and transferred to a new 15 ml tube to isolate viable protoplasts of tobacco. This tube was carefully mixed with 8 ml of a W5 solution (Table 3), treated on ice for 10 to 30 minutes, and then centrifuged at 100×g for 2 minutes. The W5 solution was removed, and then the pellet was re-diluted with a 100 μl of MMG buffer (Table 4) and the final density was adjusted to 1×10⁶ protoplasts/ml. A 100 μl of MMG-protoplast suspension was transferred to a new 15 ml round-bottom tube, and 10 μl of the protein expression module vectors (1 μg/μ1) PlantGEM-V2-1, PlantGEM-V2-5, and PlantGEM-V2-7 prepared in Example 1-1 in the protoplasts were added in the tube. 110 μl of a PEG solution (Table 5) was added and then mixed by softly tapping, incubated at room temperature (RT) for 7 minutes, and then added with 600 μl of the W5 solution and slowly inverted to stop the reaction. Thereafter, the mixture was centrifuged at 100×g for 2 minutes in a centrifuge to remove 770 μl of a supernatant, and only 50 μl of the pellet was left, and the remaining transformation-related solution and pDNA were washed. 950 μl of the W5 solution was added, incubated in the dark for 12 to 24 hours, and centrifuged at 100×g for 2 minutes at RT in a centrifuge to obtain transformed protoplasts, and transformed protoplasts were observed under a fluorescence microscope in FITC channel with 100 ms.

TABLE 2

| | | |
|---|---|---|
| Cellulase Onozuka R-10 | 250 | mg |
| Macerozyme | 50 | mg |
| K3AS | 25 | ml |
| | 25 | ml |

K3AS

| | | |
|---|---|---|
| MS salt | 0.43 | g |
| 1M CaCl₂•2H₂O (MW 147.02) | 312 | μl |
| Sucrose | 13.7 | g |

| | |
|---|---|
| DW | +ml |

| | |
|---|---|
| 100 | ml |

TABLE 3

| | | |
|---|---|---|
| 5M NaCl (MW 58.44) | 3.08 | ml |
| 1M CaCl₂•2H₂O (MW 147.02) | 12.5 | ml |
| 2M KCl (MW 74.5) | 0.535 | ml |
| Glucose | 0.109 | g |
| 0.2M MES-KOH (MW 213.2) | 1 | ml |
| DW | 82.88 | ml |
| Total | 100 | ml |

TABLE 4

| | | |
|---|---|---|
| 0.8M Mannitol (MW 132.2) | 25 | ml |
| 1M MgCl₂•6H₂O (MW 203.3) | 0.75 | ml |
| 0.2M MES-KOH (MW 213.2) | 1 | ml |

| | |
|---|---|
| DW | +ml |

| | |
|---|---|
| 50 | ml |

TABLE 5

| | | |
|---|---|---|
| PEG4000* | 4 | g |
| 0.8M Mannitol (MW 132.2) | 2.5 | ml |
| 1M CaCl₂•2H₂O (MW 147.02) | 0.2 | ml |

| | |
|---|---|
| DW | +ml |

| | |
|---|---|
| 10 | ml |

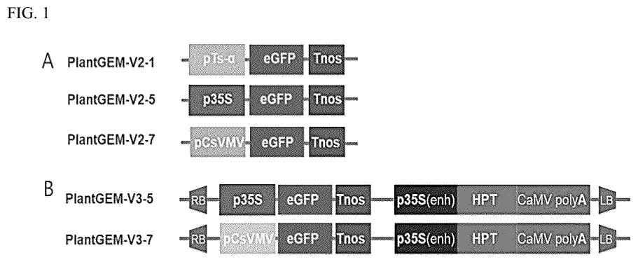

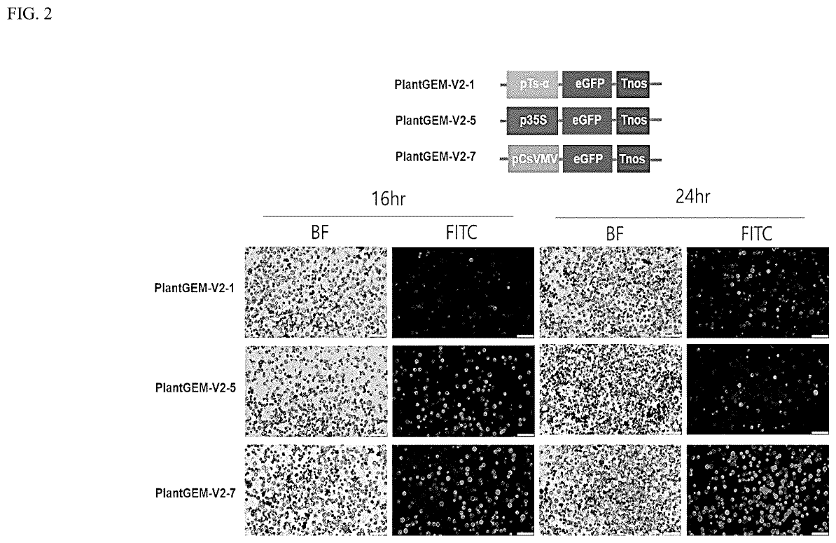

As a result, both the protein expression module vectors PlantGEM-V2-5 and PlantGEM-V2-7 were found to operate normally in tobacco protoplasts, and PlantGEM-V2-7 showed strong GFP expression compared to controls (FIG. 2). In addition, as the protoplast incubation time elapsed for 24 hours, the expression of PlantGEM-V2-5, which was regulated by CaMV35S, was decreased, whereas the fluorescence of GFP was strongly maintained in PlantGEM-V2-7, which was regulated by the CsVMV promoter. This indicated that the expression regulation power of the CsVMV promoter was stronger than that of CaMV35S or pTSα.

### Example 2. Confirmation of Protein Expression Through Radio Frequency Treatment

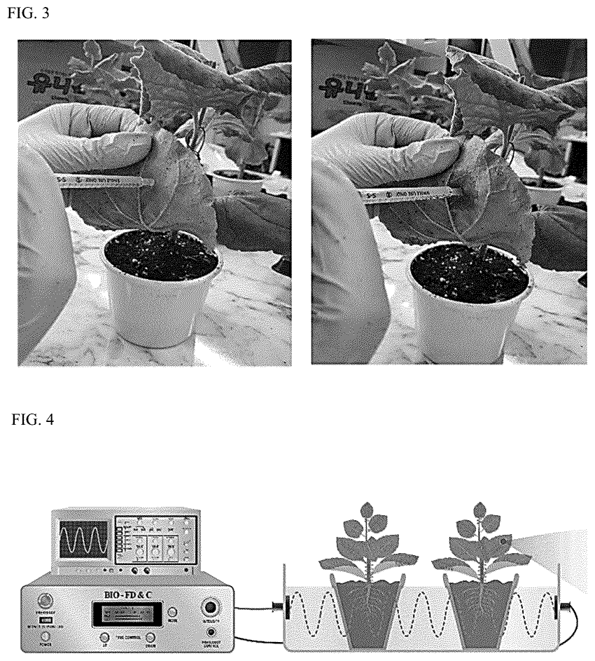

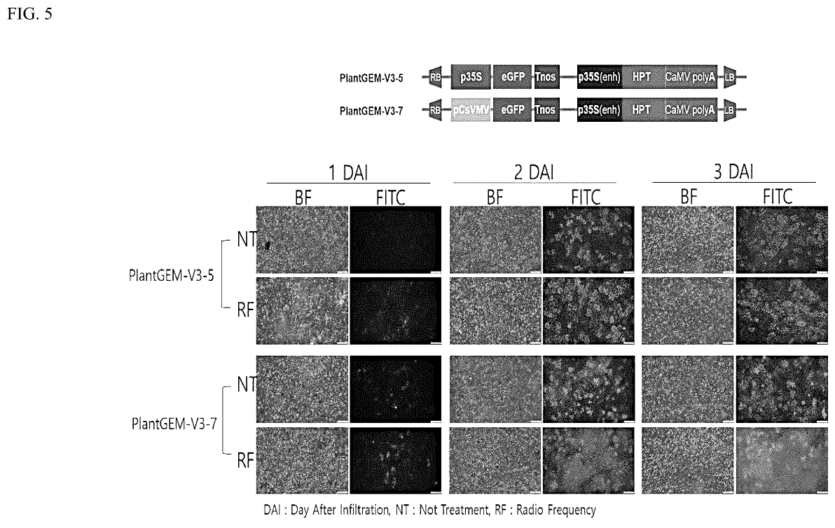

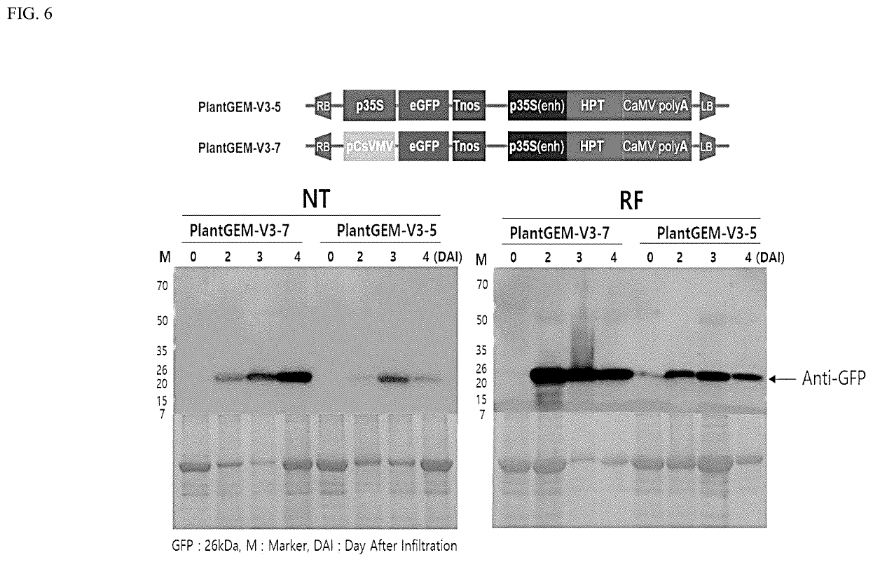

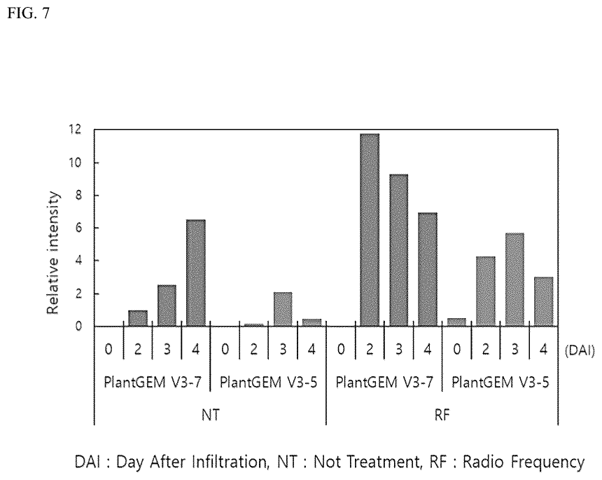

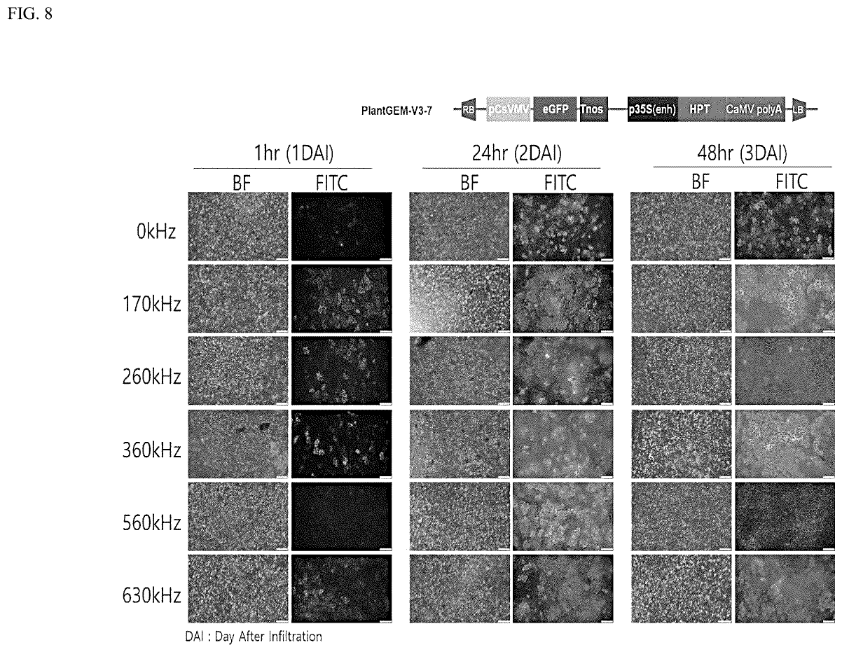

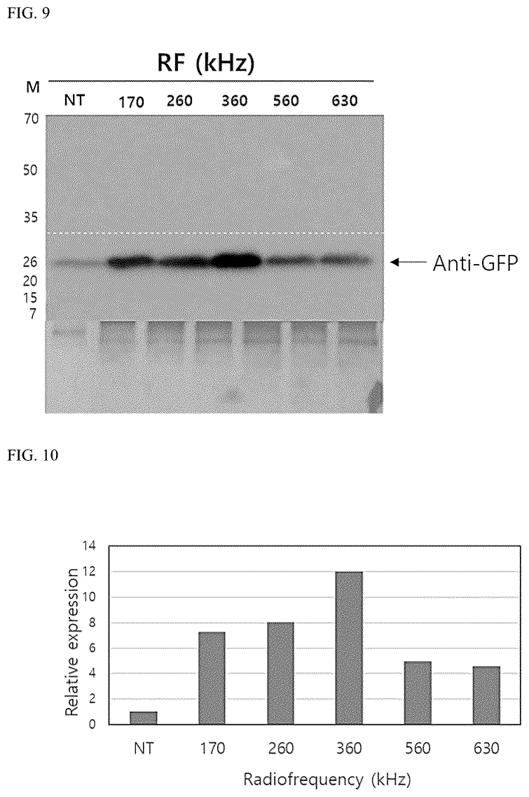

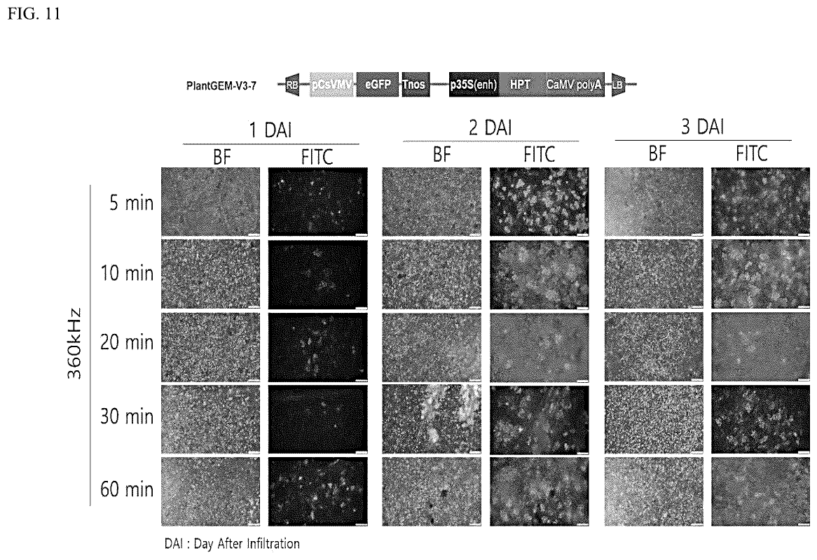

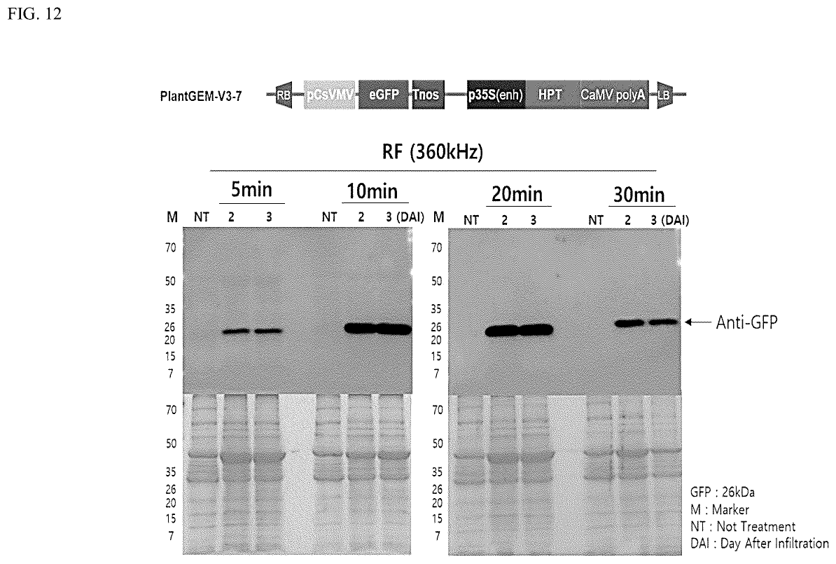

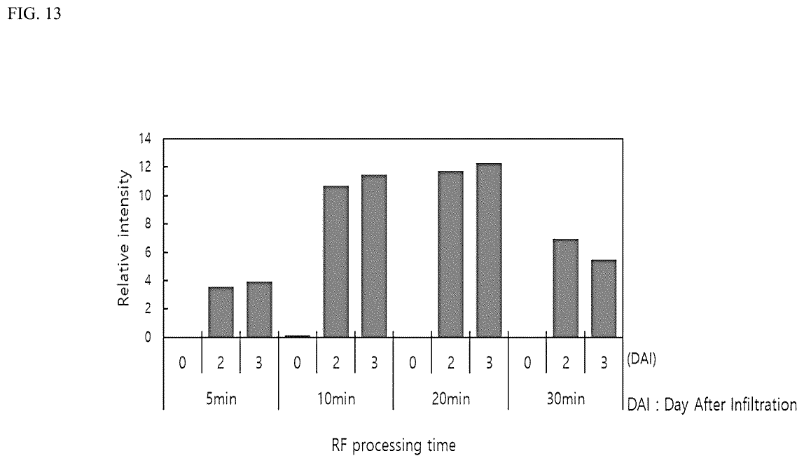

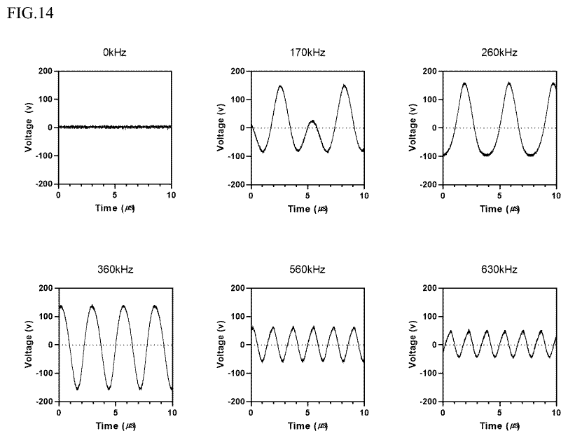

Binary vectors PlantGEM-V3-5 and PlantGEM-V3-7 consisting of the GFP protein expression module prepared in Example 1 were inoculated into plant leaves using an agroinfiltration method, and then treated with a radio frequency, and the expression of foreign protein (GFP) was confirmed. In the agroinfiltration method, the expression of foreign protein may be confirmed within a few days after inoculating the leaves of plants with Agrobacteria, and thus there is an advantage of obtaining high expression of the foreign protein in a short time. However, since various elements of an expression system act as important factors in determining the efficiency of protein expression, in the present disclosure, the radio frequency (RF) was treated at 1 day after infiltration, which was the time point at which the protein expression started, to investigate whether the CsVMV promoter responded to the radio frequency. Specifically, the binary vectors PlantGEM-V3-5 and PlantGEM-V3-7 consisting of the GFP protein expression module prepared in Example 1 were transformed into Agrobacteria, respectively, and then the Agrobacteria were line-smeared on a YEP solid medium and incubated for about 2 to 3 days in 28° C. Thereafter, one colony was picked from the culture medium, inoculated into 3 mL of a liquid YEP medium containing antibiotics, and incubated for 36 to 72 hours in a shaking incubator at 28° C. until the OD600 value reached 1.2 to 1.8. Thereafter, the Agrobacteria were diluted with an infiltration buffer solution according to the OD600 value of 0.2. Then, lower surface of the 4th to 8th leaves from the bottom of the tobacco plants was slightly wounded with a syringe needle and infiltrated the Agrobacteria solution into the leaves (syringe infiltration) (FIG. 3). After 24 hours of infiltration with Agrobacteria, a radio frequency of 360 kHz was treated using a self-made radio frequency treatment device. For the radio frequency treatment, the water tank was filled with water to immerse the plant pot and the electrodes installed on both sides of the tank. The pot in which Agro-infiltrated plant was planted was immersed in the water tank, and then treated with the radio frequency by connecting a self-made radio frequency generator to both the electrodes (FIG. 4). After 1 day, 2 days, 3 days, and 4 days of radio frequency (RF) treatment, each leaf was collected and the GFP expression was confirmed with a fluorescence microscope. To determine whether the GFP expression increased in response to RF, the expression level of GFP protein was analyzed by Western blot assay. Finally, the expression of GFP was compared with the expression pattern by a 35S promoter as a control group.

As a result, in a control group untreated with RF, PlantGEM-V3-7 began to weakly express GFP on day 2 of infiltration, and the expression continued to increase until day 4, and PlantGEM-V3-5 showed a weak GFP band on day 3 after infiltration (FIGS. 5 and 6). On the other hand, when the RF was treated on day 1 after infiltration (1 DAI), the GFP expression increased about 12-fold on day 2 showing a maximum value during an observation period, and the expression level gradually decreased on days 3 and 4 (FIGS. 5 and 6). A transient recombinant protein expression pattern, which usually showed the maximum protein expression after 4 to 5 days of infiltration, was changed by RF stimulation, and thus, in the case of PlantGEM-V3-7, a protein maximum was shown at 2 DAI by treating RF (FIG. 6). That is, in response to RF treatment, the protein expression period was shortened to 2 days after Agrobacteria inoculation, and at the same time, the protein expression level was also remarkably increased (FIG. 7).

Therefore, it was confirmed that the PlantGEM-V3-7 vector operated as an inducible module in which the protein expression was strongly induced in response to RF treatment.

### Example 3. Confirmation of Protein Expression According to RF Frequency Treatment

Since RF treatment has appeared to be a very important tool for improving the production of recombinant proteins in plants, it is necessary to clarify the optimal conditions for RF treatment to reach the maximum production of recombinant proteins in plant expression systems. Specifically, after 1 day (24 H) of infiltration with Agrobacteria carrying the PlantGEM-V3-7 vector, plants were treated once for 20 minutes at frequencies of 170, 260, 360, 560 and 630 kHz, respectively, and on day 2 (2 DAI) of infiltration, the expression level of GFP, a marker protein, was observed using a fluorescence microscope and quantitatively analyzed by Western blot assay.

As a result, it was found that the protein expression was most significantly increased at 360 kHz (FIGS. 8 to 10).

Through this, the high-efficiency treatment conditions of the vector system of the present disclosure were confirmed and can be used for transient expression and the like.

### Example 4. Confirmation of Protein Expression by RF Treatment Time

In order to derive the optimal combination between RF and target gene expression, the RF treatment time that induced the highest expression of the target protein was examined by comparing the protein expression levels depending on different RF treatment time. Specifically, tobacco was infiltrated with Agrobacteria containing the PlantGEM-V3-7 vector, and after 1 day (24 H), the plants were treated with RF at a frequency of 360 kHz for 5, 10, 20, 30, and 60 minutes, respectively, and then the expression level of GFP, a marker protein, was observed using a fluorescence microscope and quantitatively analyzed by Western blot assay.

As a result, when RF was treated for 10 or 20 minutes, the GFP protein expression was significantly increased (FIGS. 11 to 13), and the optimal RF treatment time was confirmed as 20 minutes.

Through the results, Agrobacteria including the vector of the present disclosure in plants were infiltrated in plants and then treated with RF to improve the production of the recombinant protein and thus can be used as an efficient plant-based protein production platform.

### Example 5. Confirmation of Radio-Frequency Waveforms Generated in Agroinfiltrated Tobacco Leaves During RF Treatment Based on the Processing Frequency

RFs were treated for 20 minutes each, RF waveforms determined at each treatment frequency using an oscilloscope (Tectronics 2012C) on crop-infiltrated tobacco plant leaves after setting the RF intensity to 20 J/cm 2 in a home-built RF generator.

By processing RF with different RF intensity, it was found that the wavelength became shorter and the amplitude (voltage) decreased as the frequency increased. As a result, the best RF waveform was found at 360 KHz, based on GFP protein level (FIG. 14).

When we apply a high-frequency current that repeats very short oscillations, the molecules will move in one direction and then immediately move in the opposite direction, causing mechanical oscillations in the plant. In reality, the movement of the particles is negligible, but they are momentarily moving back and forth, colliding with other adjacent areas. This subtle movement of cell components can cause physical stress on the plant cells. In response to this stress, we believe that plant cells undergo changes in protein or RNA stability and other cellular functions, which in turn alter many physiological processes in the plant, leading to increased protein expression of the target protein after RF treatment. From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

[National research and development project supporting the invention]

[Project Unique Number] 1415179433

[Project Number] 20015900 [Ministry name] Ministry of Trade and Industry and Energy

[Project Management (Special) Institution Name] Korea Evaluation Institute of Industrial Technology

[Research Project Name] Advanced industrial LMO risk assessment

[Research Subject Name] Advancement of industrial genetically modified plant cells for EGF production

[Percent Contribution] ½

[Project performance institute name] BIO-FD&C Co., Ltd.

[Research Period] Apr. 1, 2021, to Dec. 31, 2023

[National research and development project supporting the invention] [Project Unique Number] 1415181181

[Project Number] 20017936

[Ministry name] Ministry of Trade and Industry and Energy

[Project Management (Special) Institution Name] Korea Evaluation Institute of Industrial Technology [Research Project Name] Excellent Enterprise research institute foster project (ATC+)

[Research Subject Name] Development of growth factors and antibody drugs using plant cell-based platform technology and global advancement of fragrance material products.

[Percent Contribution] ½

[Project performance institute name] BIO-FD&C Co., Ltd.

[Research Period] Apr. 1, 2022, to Dec. 31, 2025

What is claimed is:

1. A promoter comprising a nucleotide sequence represented by SEQ ID NO: 1.

2. The promoter of claim 1, wherein the promoter is an inducible promoter that increases expression of a target protein by a radio frequency.

3. An expression cassette comprising the promoter of claim 1, a nucleic acid encoding a target protein, and a Nos terminator (Tnos).

4. A recombinant vector for plant expression comprising the expression cassette of claim 3.

5. The recombinant vector for plant expression of claim 4, further comprising coding sequence for plant selection marker genes.

6. The recombinant vector for plant expression of claim 4, wherein expression of a target protein is increased by radio frequency (RF) treatment.

7. The recombinant vector for plant expression of claim 4, wherein expression of a target protein is increased by radio frequency (RF) treatment of 30 KHz to 30 MHz.

8. The recombinant vector for plant expression of claim 7, wherein expression of a target protein is increased by radio frequency (RF) treatment of 360 KHz.

9. The recombinant vector for plant expression of claim 4, wherein the plants are plant cells, plant cultured roots, or plant bodies.

10. The recombinant vector for plant expression of claim 4, further comprising:
a fluorescent material.

11. The recombinant vector for plant expression of claim 10, wherein the fluorescent material is fluorescent protein, photoprotein, luciferase, fluorescent dye or time-resolved fluorescence (TRF).

12. Agrobacteria transformed with the recombinant vector for plant expression of claim 4.

13. A plant transformant introduced with the Agrobacteria of claim 12 to produce a target protein.

14. The plant transformant of claim 13, wherein the transformant is plant cultured cells, plant cultured roots, or plant bodies.

15. A method for producing a target protein comprising:
a) preparing a transient or stable plant transformants expressing a target protein by introducing the vector of claim 4 into a plant;
b) treating a radio frequency; and
c) isolating and purifying the target protein from the transformant.

16. The method for producing the target protein of claim 15, wherein in step a), the plant transformant is prepared by transforming the vector into the plant.

17. The method for producing the target protein of claim 15, wherein in step a), the plant transformant is prepared by infiltrating Agrobacteria transformed with the recombinant vector into the plant.

18. The method for producing the target protein of claim 15, wherein a radio frequency of 30 KHz to 30 MHz is treated.

19. The method for producing the target protein of claim 15, wherein the radio frequency is treated for 1 to 120 minutes.

20. The method for producing the target protein of claim 19, wherein the radio frequency is treated for 20 minutes.


SEQUENCE LISTING (5 sequences)

SEQ ID NO: 1 - DNA, 508 bp, synthetic construct
  source 1..508: mol_type = other DNA
ccagaaggtaattatccaagatgtagcatcaagaatccaatgtttacgggaaaaactatggaagtattatgtgagctcagcaagaagcagatcaatatgcggcacatatgcaacctatgttcaaaaatgaagaatgtacagatacaagatcctatactgccagaatacgaagaagaatacgtagaaattgaaaaagaagaaccaggcgaagaaaagaatcttgaagacgtaagcactgacgacaacaatgaaaagaagaagataaggtcggtgattgtgaaagagacatagaggacacatgtaaggtggaaaatgtaagggcggaaagtaaccttatcacaaaggaatcttatcccccactacttatccttttatatttttccgtgtcatttttgcccttgagttttcctatataaggaaccaagttcggcatttgtgaaaacaagaaaaaatttggtgtaagctattttctttgaagtactgaggatacaacttcagagaaatttgt

SEQ ID NO: 2 - DNA, 253 bp, synthetic construct
  source 1..253: mol_type = other DNA
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 3 - DNA, 346 bp, synthetic construct
  source 1..346: mol_type = other DNA
tgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaaca

SEQ ID NO: 4 - DNA, 1026 bp, synthetic construct
  source 1..1026: mol_type = other DNA
atgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatag

SEQ ID NO: 5 - DNA, 175 bp, synthetic construct
  source 1..175: mol_type = other DNA
tttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatc